(19) United States
(12) Patent Application Publication
(10) Pub. No.: US 2003/0187498 A1
(43) Pub. Date: Oct. 2, 2003

(54) Chamfered stent strut and method of making same

(75) Inventor: Gordon B. Bishop, Windsor, CA (US)

Correspondence Address: MEDTRONIC AVE, INC., 3576 UNOCAL PLACE, SANTA ROSA, CA, 95403, US

(73) Assignee: Medtronic AVE, Inc.

(21) Appl. No.: 10/107,473

(22) Filed: Mar. 28, 2002

(51) Int. Cl. A61F 2/06

(52) U.S. Cl. 623/1.16; 216/10

(57) ABSTRACT

A prosthesis for implantation in a blood vessel includes struts with a cross-section having a flat base with angled edges on the inner surface and chamfered edges on the outer or exterior surface. The base angled edge is substantially a right angle, while the outer or exterior surface has edges that are chamfered with either round or angled chamfers. The chamfers could be manufactured by either machining the chamfer into the stock material, or by tumbling the stent in a tumbler to abrasively wear the edges to form a chamfer.

### BACKGROUND OF THE INVENTION

1. Field of the Invention

This invention relates generally to a medical device. More specifically, the invention relates to an implantable stent prosthesis for treatment of stenosis in blood vessels.

2. Background Art

Cardiovascular disease, including atherosclerosis, is the leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA”. The objective in angioplasty is to enlarge the lumen of the affected coronary artery by radial hydraulic expansion. The procedure is accomplished by inflating a balloon within the narrowed lumen of the coronary artery. Radial expansion of the coronary artery occurs in several different dimensions, and is related to the nature of the plaque. Soft, fatty plaque deposits are flattened by the balloon, while hardened deposits are cracked and split to enlarge the lumen. The wall of the artery itself is also stretched when the balloon is inflated.

Unfortunately, while the affected artery can be enlarged, in some instances the vessel restenoses chronically, or closes down acutely, negating the positive effect of the angioplasty procedure. In the past, such restenosis has frequently necessitated repeat angioplasty or open heart surgery. While such restenosis does not occur in the majority of cases, it occurs frequently enough that such complications comprise a significant percentage of the overall failures of the angioplasty procedure.

To lessen the risk of restenosis, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent overcomes the natural tendency of the vessel walls of some patients to restenoses, thus maintaining the patency of the vessel.

Stents are delivered to the lesion, or target area, by a catheter device. Typically, the stent is introduced to the patient in an unexpanded form, having the smallest diameter possible. The small diameter is necessary during insertion in order to properly traverse tortuous blood vessels. When the stent reaches the target area, the stent is expanded to engage the blood vessel walls, enlarging the inner circumference of the blood vessel, and securing to vessel wall. When the stent is positioned across the target area, it is expanded, causing the length of the stent to contract and the diameter to expand.

The stent may be expanded by a number of methods, including expansion of the stent using a balloon on a balloon catheter. The balloon is inserted into the unexpanded stent, either before insertion to the patient or after the stent has reached the target site. The balloon is inflated while inside the circumference of the stent, forcing the stent to expand and lodge within the blood vessel at the target site.

Stents are generally formed using any of a number of different methods. One group of stents are formed by winding a wire around a mandrel, welding or otherwise forming the stent to a desired configuration, and finally compressing the stent to an unexpanded diameter. Another group of stents are manufactured by machining tubing or solid stock material into bands, and then deforming the bands to a desired configuration. Another group of stents are formed by laser etching or chemical etching, which cuts or etches a tube to a desired shape. The stent is usually etched or cut in a unexpanded state.

A drawback of previously known stents manufactured using standard procedures, however, is that the outside surface of the stent struts, which contacts the blood vessel when implanted, has the same configuration and shape as the inside surface of the stent struts, which contacts the balloon used to expand the stent. When the stent is formed from a wire, the stent struts have rounded edges, as would be expected from a wire or ring. Rounded edges that contact the outer surface of the balloon, however, are smooth and may allow the stent to slip or slide along the balloon during insertion through a blood vessel. Furthermore, in the event that a physician determines that the lesion to be treated cannot be accessed with the catheter and stent because the vessel is too narrow or too occluded, the stent must be withdrawn with the catheter. In this situation, the rounded edges of the stent against the balloon may allow the stent to move longitudinally on the balloon at the distal end of the catheter as the catheter and stent are withdrawn through the vasculature. As a worst case scenario, it may result in the stent completely sliding off the balloon, leaving the unexpanded stent within a blood vessel. Additionally, even after successfully reaching a target site within the patient's vasculature, the stent can slide and slip on the balloon during expansion of the balloon, which may result in a sub-optimal asymmetric deployment and positioning of the stent at the target site. A variety of techniques are used to keep stents from moving longitudinally on the balloon. These focus on the interface of the stent and the delivery balloon.

When the stent is formed by machining, etching or cutting, the stent struts have 90 degree angles, and are either square or rectangular shaped in cross-section. Stents with square or rectangular edges, however, have decreased trackability when they are inserted through a patients vasculature and result in greater vessel trauma when implanted.

It would therefore be desirable to provide a stent and method, useful for treating chronic restenosis conditions, that avoids the problem of compromising the effectiveness of either the stent against the vessel wall or the stent against the balloon.

### BRIEF SUMMARY OF THE INVENTION

The present invention is a prosthesis for implantation in a blood vessel having struts with a cross-section including a flat base with angled edges on the inner surface and chamfered edges on the outer or exterior surface. The angled edge of the base is substantially a right angle, but may be an angle that extends slightly greater than 90 degrees to substantially less than 90 degrees.

The struts include an exterior surface having edges that are either round chamfers or angled chamfers. The round chamfer may be either a single smooth curve, such as a D-shape, or may have chamfers having a radius R. If the exterior surface of the struts includes an angled chamfer, the angle chamfer is preferably between about 30 and 60 degrees, and more preferably about 45 degrees.

One method of manufacturing the prosthesis of the invention is machining the prosthesis from stock material. The edges of the exterior surface of the proximal end of the material is chamfered with either an angled or round chamfer. A second opposing chamfer is formed into the exterior of the material at an appropriate distance distal of the proximal end of the cylinder. The end of the stock material having the chamfers is parted just distal of the second chamfer, to form a ring having an exterior surface with chamfered edges. This forms a stent segment which can then be attached to other stent segments for the desired segment length.

Another method of manufacturing the prosthesis of the invention is forming the prosthesis using standard machining or etching processes, placing the prosthesis around a mandrel with a close fit, and placing the prosthesis and mandrel within an abrasive tumbler. The prosthesis and mandrel are tumbled until a rounded edge is worn onto the exterior surface edges of the prosthesis. The inner surface edges remain at substantially right angles because they are protected from abrasive wear by being in direct contact with and extending around the mandrel.

### BRIEF DESCRIPTION OF THE DRAWINGS/FIGURES

The foregoing and other features and advantages of the invention will be apparent from the following, more particular description of preferred embodiments of the invention, as illustrated in the accompanying drawings.

FIG. 1 is view of an implantable endoprosthetic stent on a balloon of a balloon catheter in an expanded configuration.

FIG. 2 is a cross-section of FIG. 1 along lines 2-2.

FIG. 3 is an enlarged cross-section of a strut of the present invention shown in FIG. 2.

FIG. 4 is a cross-section of another embodiment of a strut of the present invention.

FIG. 5 is a modular stent in an unrolled form.

FIG. 6 is a tubular slotted stent which may include the struts of the present invention.

### DETAILED DESCRIPTION OF THE INVENTION

The preferred embodiments of the present invention are now described with reference to the figures where like reference numbers indicate identical or functionally similar elements. While specific materials and method steps are discussed, it should be understood that this is done for illustrative purposes only. A person skilled in the relevant art will recognize that other materials or method steps can be used.

Generally, the stent of the present invention is designed to be placed in a patient's blood vessel using a balloon catheter. The stent may be disposed on the balloon and introduced through a patient's vasculature to a treatment site, or may placed at the treatment site, and only expanded using a balloon of a balloon catheter. The stent has an inner circumference that includes edges that are angled at a right angle to increase the coefficient of friction of the balloon against the stent edges. Such a configuration aids in introducing or tracking the stent through a vasculature because it reduces the chance that the stent will slip or slide either along the balloon or completely off the balloon. This configuration further aids during expansion of the stent by allowing the balloon to expand about the edges to apply forces against the stent in directions not normal to the stent struts.

The stent has chamfered surfaces on the outer circumferential edges. The chamfered surface increases the trackability which aids in placement of the stent by enabling the stent to more easily and smoothly move through a vasculature. The chamfered edge aids in introducing the stent into a body through a blood vessel because the edge slides over obstacles instead of engaging them on a sharp edge. This is especially important when a stent has been previously placed at one location, and the vessel now needs to be treated at a location distal of the placed stent. The new stent must pass through the old stent without catching on, or dislodging the old stent.

The chamfered edge also aids in trackability by reducing the force required by the physician introducing the stent. Because the chamfered edge is less inclined to catch on obstacles, and can more easily be introduced through narrow regions, less force is required to introduce then stent, as well as less twisting and working of the catheter and stent assembly.

FIG. 1 shows a stent-catheter assembly 100 including a sinusoidal modular stent 102 disposed about a balloon 104 on a balloon catheter. The balloon catheter includes balloon 104 and catheter shaft 106. Balloon 104 is an expanded configuration, as it would be if it were in the process of expanding stent 102 into a blood vessel.

Stent 102 may be made of any suitable biocompatible material such as stainless steel, titanium, tantalum, superelastic NiTi alloys or high strength thermoplastic polymers. The diameter of stent 102 is very small, so the tubing from which it is made must necessarily also have a small diameter. Typically stent 102 has an outer diameter of about 0.06 inch (0.15 cm) in the unexpanded condition, and can be expanded to an outer diameter of 0.1 inch (0.25 cm) or more. The wall thickness of stent 102 is about 0.003 inch (0.0076 cm).

Stent 102 includes struts 108 and crowns 110. Crowns 110 are the bends in stent 102, and struts 108 are the bars extending between crowns 110. Crowns 110 need not be bends or curves, but could be cross-bars or connectors that connect struts 110 together. Likewise struts 108 need not be straight, but may be curved or designed elements that extend between crowns 110.

FIG. 2 is a cross-section of FIG. 1 taken along lines 2-2. FIG. 2 shows balloon 104 and struts 108. Struts 108 include a base 206 and a top surface 208. Base 206 includes the surface that comprises the inner diameter of stent 102, which is also the surface of strut 108 that faces inwardly, toward the center of balloon 104. Top surface 208 of strut 108 includes the surface that is substantially facing away from the center of balloon 104, or may be considered the surface that comprises the outer diameter of strut 108.

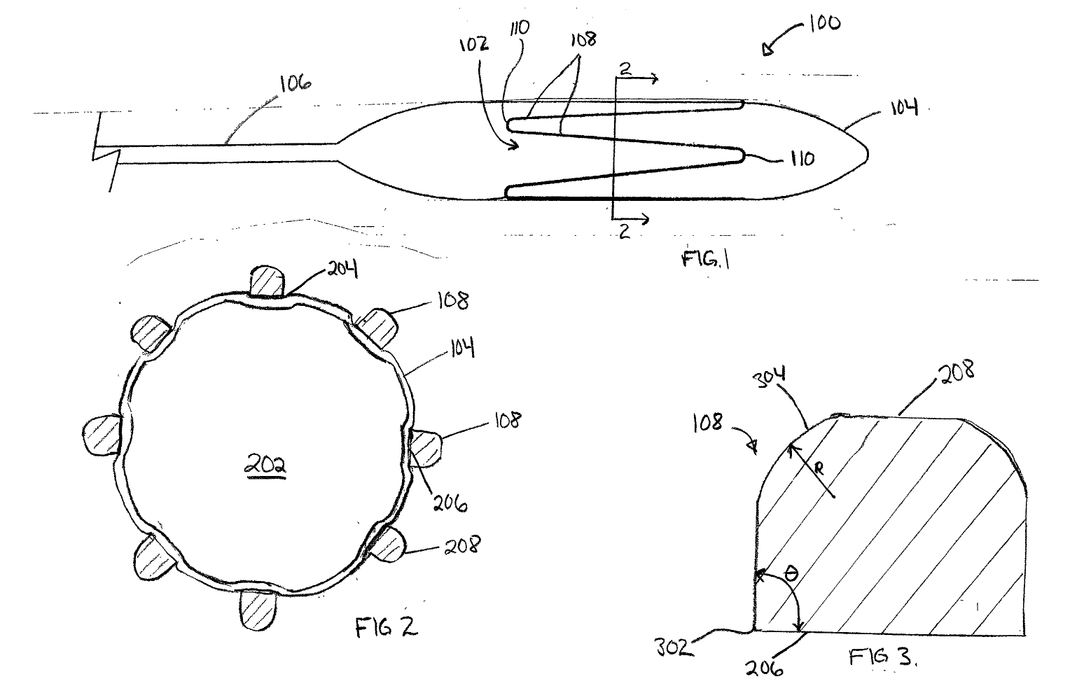

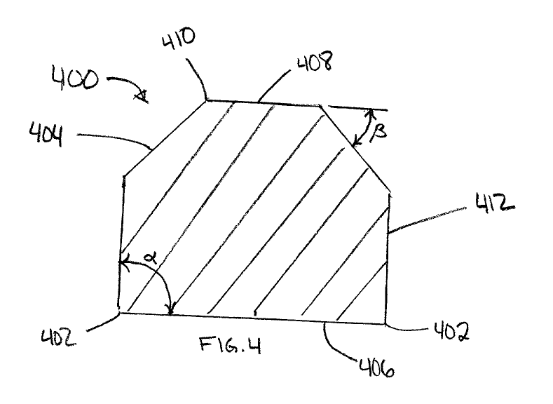

A cross-section of strut 108 is more clearly seen in FIG. 3. As shown in FIG. 3, base 206 is substantially flat, and includes an angled edge 302, with the angle designated by θ. The angle θ is preferably substantially a right angle, but may be an angle that extends slightly greater than 90 degrees to substantially less than 90 degrees.

As shown in FIG. 3, top surface 208 includes the surface facing opposite that of base 206. Top surface 208 does not have angled edges, but includes rounded edges 304. Rounded edges 304 are round chamfers on struts 108 that enable better trackability of stent 102 during insertion and placement of the stent in a patient's vasculature. Additionally, it is rounded edges 304 that contact and frictionally secure stent 102 to the blood vessel walls. Rounded edges 304 inflict less vessel trauma than stents having square ends. The size of rounded edges 304 can vary in radius, designated by R. Preferably, rounded edges 304 have a radius of about 0.0005 to 0.003 inch (0.00127 to 0.0076 cm), and more preferably have a radius of about 0.001 inch (0.0025 cm).

In one embodiment, radius R is one-half of the width of base 206. As such, the cross-section of strut 108 is a D-shaped strut, having a smooth curve completely across top surface 208. However, as would be apparent to one skilled in the art, radius R could be smaller than one-half of the width of base 206 at each rounded edge 304 or could be larger. Furthermore, the radius R of the two rounded edges 304 need not have the same radius, but could be larger or smaller, if preferred.

In a preferred embodiment, crowns 110 also have the cross section described and shown with reference to FIG. 3. However, as would be apparent to one skilled in the relevant art, crowns 110 need not match struts 108 in cross-section.

Returning now to FIG. 2, balloon 104 includes a chamber which is filled with an inflation fluid during inflation. Balloon 104 is formed of a thin pliable material capable of expanding from a small unexpanded, collapsed state for introducing the balloon into a blood vessel to a large expanded diameter. Balloon 104 could be a balloon formed from polyethylene terephthalate (PET) in a drawing and blow molding process so as to provide biaxial orientation to the material. PET balloons are found to exhibit the desirable properties of high burst strength and relatively low radial expansion when inflated to high pressures. Alternatively, balloon 104 could be formed from polyethylene, PVC, polypropylene, nylon or other material, as would be apparent to one skilled in the relevant art.

During insertion of a stent and a balloon through a patient's vasculature, the friction between the exterior of the stent and the blood vessel wall may cause the stent to slip along the outer surface of the balloon, until the stent is no longer properly located at the center of the balloon. This is especially common when the stent is being introduced through a narrow region of a blood vessel. Occasionally, during introduction of the stent, the physician may determine that the lesion to be treated cannot be accessed by the catheter and stent because the vessel is too narrow. The physician must then attempt to withdraw the catheter and stent from the patient's body. In this situation, there is some chance that the stent may slip off the balloon at the distal end of the catheter in an unexpanded state, leaving the unexpanded stent within a blood vessel.

Because stent 102 of the present invention includes base 206 having angle 302 which, as explained above, is slightly greater than 90 degrees to substantially less than 90 degrees, the sharp edge of strut 108 digs into or grabs the unexpanded surface of balloon 104 while advancing or retracting stent 102 through a patient's vasculature.

After reaching the target site and during expansion, balloon 104 exerts force against base 206 of struts 110. However, because balloon 104 is formed from a thin pliable material, pressure exerted against the exterior of balloon 104 during expansion causes deformation of the exterior of balloon as shown at 204. Accordingly, pliable balloon 104 expands slightly around strut 108. Because base 206 includes angle 302 which, as explained above, is slightly greater than 90 degrees to substantially less than 90 degrees, strut 108 does not easily slide over the surface of balloon 104. Accordingly, strut 108 is held in place during expansion. This aids the physician in properly placing the stet in a patient because the stent will not migrate or slip on the balloon, displacing the stent from its desired position during placement.

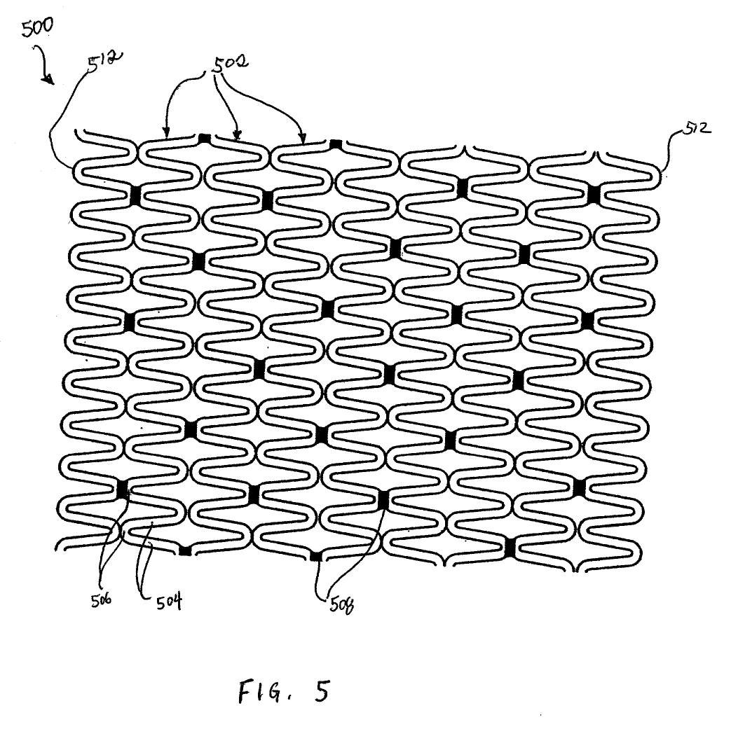

FIG. 4 shows a second embodiment of a cross-section of a strut 400 of the present invention. Strut 400, includes a base 406 and a top surface 408. Base 406 is the surface that forms the inner circumference of a stent, and is the surface that directly contacts the balloon when implanted in a patient. Top surface 408 is the surface that forms the outer circumference of the stent, and is the surface that directly contacts a blood vessel wall when placed in a patient. Base 406 of strut 400 includes angled edge 402, with the angle designated by α. The angle α is preferably substantially a right angle, but may be an angle that extends slightly greater than 90 degrees to substantially less than 90 degrees.

Strut 400 also includes a chamfered surface 404. Unlike the rounded chamfer of FIG. 3, chamfered surface 404 creates an angled surface, forming an angled intersection at the point that top surface 408 and chamfered surface 404 meet, as shown at 410. Chamfered surface 404 is angled from top surface by an angle designated as β. The angle β is preferably between about 30 and 60 degrees, and more preferably about 45 degrees. If angle β is greater than about 60 degrees, the intersection 410 becomes more pointed, increasing the incidence of vessel trauma when implanted. Vessel trauma increases as angle P increases. Likewise, when angle β approaches a very small angle, the benefit of chamfer 404 decreases because chamfer 404 begins to merge with top surface 408, and a side 412 of strut 400 extends nearly to the top surface 408, and forms an edge that may irritate the blood vessel.

In another embodiment of the strut of FIG. 4, chamfer 404 is not a single chamfer, but is multiple chamfers that effectively form a curve from top surface 408 to side 412. The multiple chamfers could be a first chamfer at 30 degrees and a second chamfer at 60 degrees. The multiple chamfers could be any number of chamfers. Naturally, the more chamfers included on the strut, the more the chamfer will appear to be a curve.

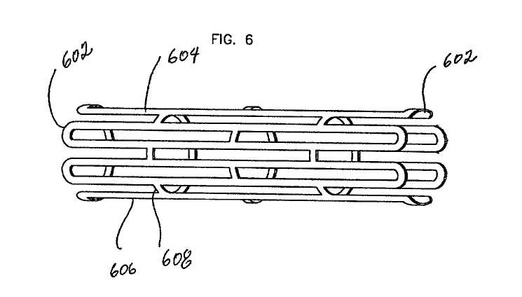

FIG. 5 shows a modular stent 500 with struts that could have the angles and curves of the present invention. Stent 500 is shown in an unrolled configuration. Stent 500 includes sinusoidal-shaped modules 502 comprising struts 504 and crowns 506. Crowns 506 are the bends or connectors that link struts 504. Stent 500 includes leading and trailing edges 512. Leading and trailing edges 512 are interchangeable, as they are determined only by which end is inserted into a patient first.

In this embodiment, modules 502 are joined together at attachment points 508. Preferably, attachment points 508 are welded joints that connect modules 502. However, attachment points could be accomplished by brazing, soldering, or other attachment methods as would be apparent to one skilled in the relevant art. In one embodiment, modules 502 are formed integral with one another.

In a preferred embodiment, both struts 504 and crowns 506 have the cross-sectional configuration described with reference to FIGS. 3 or 4. However, in one embodiment, only the struts have the cross-sectional configuration described with reference to FIGS. 3 and 4.

Alternatively, only the leading and trailing edges, designated as 512, of stent 500 have the cross-sectional configuration described with reference to FIGS. 3 and 4, and all other struts and crowns have standard square or rectangular cross-sectional configurations. Chamfering the leading edges aids in tracking the stent through a vasculature prior to placement.

FIG. 6 shows a tubular slotted stent which may implement the chamfered edges of the present invention. Tubular-slotted stents are one type of implantable stent which involve what may be thought of as a tube having a number of slots cut in its wall, resulting in a mesh when expanded. A tubular-slotted stent is typically cut out of a hypo-tube, or out of a sheet, which is then rolled, and welded to form a the tube. Example of such stents include, but are not limited to, those disclosed in U.S. Pat. No. 5,810,868 to Lashinski et al., U.S. Pat. Nos. 4,733,665, 4,776,337, 4,739,762 and 5,102,417 all issued to Palmaz, U.S. Pat. No. 5,195,984 issued to Schatz, U.S. Pat. No. 5,421,955 issued to Lau et al., or U.S. Pat. No. 5,449,373 issued to Pinchasik et al.

FIG. 6 is an example of a tubular-slotted stent and is included only to signify that the struts of the present invention, having the cross-section of FIGS. 3 or 4, may be used on tubular slotted stents. Accordingly, for ease of description, FIG. 6 is described together with like elements having like numbers. Further, as described with respect to FIG. 5, a plurality of the tubular-slotted stent segments shown in FIG. 6 may be linked together to form the desired stent length.

As shown in FIG. 6, the expandable intraluminal stent 600 is generally a tubular shaped member having a top or exterior surface 604 disposed between leading and trailing ends 602. Exterior surface 604 is the surface that contacts the blood vessel when stent 600 is introduced to and expanded within a blood vessel. Exterior surface 604 is formed by struts 606 intersecting with, and connected by crowns 608. Crowns 608 are cross-bars that connect struts 606 together. The edges of exterior surface 604 of struts 606 are chamfered as described with reference to either FIGS. 3 or 4. The interior surface of struts 606 are angled at substantially a right angle or less, as is described with reference to FIGS. 3 or 4.

The chamfered struts of the present invention may be manufactured by a number of different methods. One method includes placing solid stock material into a rotatable collet fixture of a machine-controlled apparatus, such as a CNC (Computer Numerical Control) lathe. The solid stock material has a distal and a proximal end, with the distal end being within the collet and the proximal end extending outward.

Once the stock material is secured within the lathe, the proximal end of the solid stock material is bored-out to create a cylinder, having an interior surface and an exterior surface. Naturally, a tube could be placed in the collet in place of the solid-stock, eliminating the need to bore the material. Preferably, the cylinder is very thin, having a thickness in the range of 0.002″ to 0.010″. However, the cylinder could be thicker or thinner, as would be apparent to one skilled in the relevant art. The exterior edge of the proximal end of the cylinder is chamfered with either an angle or round chamfer.

A second opposing chamfer is formed into the exterior of the cylinder by touching a tool to the exterior surface of the cylinder at an appropriate distance distal of the proximal end of the cylinder. The chamfer is formed into the exterior surface of the cylinder, with the deepest cutting along the edge that will become the distal side of a ring when parted from the cylinder, as will be explained below. The distance will be dependent on the desired width of the strut and the desired type of chamfer, including the angle or the radius of the chamfer desired. The second chamfer may be rounded or angled, as would be apparent to one skilled in the art, but preferably is formed to match the chamfer machined on the proximal end of the cylinder.

Once the appropriate second chamfer is formed, the proximal end of the cylinder is parted from the body of the cylinder at a point just distal of the chamfer, forming a ring. The resulting ring has a flat interior surface and an exterior surface bearing chamfered edges.

The ring is placed within a die having a top and a bottom half, with each half having extremities that form peaks and valleys. When placed together, the peaks of the top die intermesh with the valleys of the bottom die, and the peaks of the bottom die intermesh with the valleys of the top die. The top and bottom dies are brought together, deforming the ring into a sinusoidal-shaped module, having struts and crowns. In a preferred embodiment, the sinusoidal shaped stent includes between 10 and 20 struts and crowns. However, more or less crowns could be formed by varying the die shapes, as would be apparent to one skilled in the relevant art.

Depending on the length of the struts between the crowns, the module may be used alone as a sinusoidal stent, or may be welded to or otherwise connected to additional modules to form a modular stent. The resulting stent includes a cross-section having inner-diameter edges substantially at right angles, and chamfered outer diameter edges, such as are described with reference to FIGS. 3 and 4.

Another method of forming tubular slotted stents with the chamfered struts of the present invention includes placing a hypo-tube into a rotatable collet fixture of a machine-controlled apparatus, such as CNC device. The hypo-tube has a distal and a proximal end, with the distal end being within the collet and the proximal end extending outward. The hypo-tube is positioned relative to a laser, and the position of the tube is controlled by machine encoded instructions. The hypo-tube is rotated and moved longitudinally relative to the laser, which is also machine controlled. The laser selectively removes the material from the tubing by ablation, and a pattern is cut into the tube. The tube may be cut into the discrete pattern of the finished stent. The stents of FIGS. 6 and 7 are examples of stents that may be manufactured using this method. Cutting a fine structure (0.0035″ web width) requires minimal heat input and the ability to manipulate the tube with precision. It is also necessary to support the tube yet not allow the stent structure to distort during the cutting operation. In order to successfully achieve the desired end results, the entire system must be configured very carefully. The hypo-tubes typically begin with an outside diameter of 0.060″ to 0.066″ and a wall thickness of 0.002″ to 0.004″. Due to the thin-wall and the small geometry of the stent struts (0.0035″ typical strut width), it is necessary to have very precise control of the laser, its power level, the focused spot size, and the precise positioning of the laser cutting path.

The resulting tubular slotted stents will have struts with substantially right angles on both the inner diameter surface and the exterior diameter surface. To form the chamfer, the stents are placed around a mandrel, and the stents and mandrel are placed within an abrasive tumbler and tumbled until a rounded edge is worn onto the outer diameter edges of the stents. The stents may be held on the mandrel by a friction fit, or may be secured on the mandrel by capping the ends of the mandrel so that the stents cannot slide off. The inner diameter edges remain at substantially right angles because they are protected from abrasive wear by being in direct contact with and extend around the mandrel. This method produces only rounded chamfers on the stents. In one embodiment, a Model 410 tumbler machine using NSG 500 media, both manufactured by Dreher Corporation, of North Attleboro, Mass. is used to manufacture the chamfered edges of the present invention.

The radius of the chamfers is dictated by the length of time spent in the tumbler. A short amount of time in a tumbler results in a round chamfer with a small radius, while a longer amount of time in a tumbler results in a round chamfer with a larger radius.

Naturally, the tumbling method could be used on a stent cut using a lathe, so long as a mandrel or other means is used to protect the inner-diameter edges from wearing down.

One alternative to tumbling is to blast the outer-diameter edges of the stent using an abrasive grit blaster. This method again requires that the inner diameter edges be protected from wear. The blasting method allows increased control over which edges are to be rounded.

The stents of the present invention could also be formed by chemical etching by coating stainless steel hypo-tubing with a material resistant to chemical etching, and then removing portions of the coating to expose portions of underlying tubing which are to be removed to develop the desired stent structure. The exposed portions of the tubing are removed by chemically etching from the tubing exterior leaving the coated portion of the tubing material in the desired pattern of the stent structure. The etching process develops smooth openings in the tubing wall without burrs that may sometimes occur when using mechanical or laser processes. The stents are then placed on mandrels and either tumbled or blasted, as described above, to form the rounded chamfers.

One advantage to manufacturing stents using lasers and etching processes is that a plurality of stents can be formed from one length of hypo-tubing by repeating the stent pattern and providing small webs or tabs to interconnect the stents. After the laser or etching process, or after the blasting or tumbling, the stents can be separated by severing the small webs or tabs which connect them.

While the invention has been particularly shown and described with reference to preferred embodiments thereof, it will be understood by those skilled in the art that various changes in form and detail may be made therein without departing from the spirit and scope of the invention.

What is claimed is:

1. An implantable prosthesis comprising:
struts; and
crowns connecting said struts, said struts having an interior surface and an exterior surface, wherein edges of said interior surface comprise substantially right angles, and wherein edges of said exterior surface are chamfered.

2. The implantable prosthesis of claim 1, wherein said chamfers are round chamfers.

3. The implantable prosthesis of claim 2, wherein said struts have a D-shaped cross-section.

4. The implantable prosthesis of claim 2, wherein said round chamfers have a radius about in the range of about 0.0005 to 0.003 inch.

5. The implantable prosthesis of claim 2, wherein said round chamfers have a radius about in the range of 0.001 inch.

6. The implantable device of claim 1, wherein said crowns include a crown interior surface and a crown exterior surface, wherein edges of said crown interior surface comprise angles, and wherein edges of said crown exterior surface are chamfered.

7. The implantable prosthesis of claim 1, wherein said chamfers are about in the range of 30-60 degrees.

8. The implantable prosthesis of claim 1, wherein the prosthesis is a single module prosthesis.

9. The implantable prosthesis of claim 1, wherein the prosthesis is a multiple module prosthesis, and the multiple modules are fixed together.

10. The implantable prosthesis of claim 9, wherein the multiple modules are fixed together by welds.

11. The implantable prosthesis of claim 1, wherein the prosthesis is a modular stent having a sinusoidal pattern, wherein said crowns connect said struts.

12. The implantable prosthesis of claim 11 wherein said modular stent comprises between 8 and 20 struts and crowns.

13. The implantable prosthesis of claim 1, wherein the prosthesis is a tubular slotted stent.

14. The implantable prosthesis of claim 1, wherein said crowns and struts form a leading edge, and only said exterior surface of said leading edge is chamfered.

15. The implantable prosthesis of claim 1, wherein said crowns have an interior surface and an exterior surface, and wherein edges of said interior surface of said crowns comprise substantially right angles, and wherein edges of said exterior surface of said crowns are chamfered.

16. A method of making an implantable prosthesis comprising the steps of:
a.) cutting a first chamfer on an outer diameter edge of a proximal end of a cylinder;
b.) cutting a second chamfer in the outer diameter of said cylinder at a location distal of said first chamfer;
c.) parting a ring from said cylinder, said ring including said first and said second chamfers; and
d.) bending said ring to form a sinusoidal module having struts and crowns.

17. The method of claim 16, further comprising a step of boring solid stock material to form said cylinder.

18. The method of claim 16, wherein said first and said second chamfer are rounded chamfers.

19. The method of claim 16, wherein each of said steps of cutting chamfers include cutting multiple chamfers.

20. A method of making an implantable prosthesis comprising the steps of:
a.) laser-cutting or etching the prosthesis from a tube of material;
b.) placing said prosthesis around a mandrel;
c.) tumbling said prosthesis and said mandrel in a tumbler containing abrasive material to wear and round the exterior edges of said prosthesis; and
d.) removing said prosthesis from the mandrel.

21. The method of claim 20, wherein step c.) includes securing said prosthesis around said mandrel by means of a friction fit.
